(19) United States
(12) Patent Application Publication
(10) Pub. No.: US 2024/0409888 A1
(43) Pub. Date: Dec. 12, 2024

(54) METHOD FOR CULTURING AND EXPANDING NUCLEUS PULPOSUS CELLS DERIVED FROM INTERVERTEBRAL DISCS AND FOR IMPROVING LOWER BACK PAIN USING NUCLEUS PULPOSUS CELLS

(71) Applicant: RegenCELLS Biomedical Co. Ltd., Taipei City (TW)

(72) Inventors: Shih-Chen TSAI, New Taipei City (TW); Wei-Hung CHEN, New Taipei City (TW)

(73) Assignee: RegenCELLS Biomedical Co. Ltd., Taipei City (TW)

(21) Appl. No.: 18/494,879

(22) Filed: Oct. 26, 2023

(30) Foreign Application Priority Data
Jun. 7, 2023 (TW) 112121278

(51) Int. Cl. C12N 5/079; A61K 35/30; C12N 9/64; C12N 9/76; C12Q 1/686

(52) U.S. Cl. CPC C12N 5/0618; A61K 35/30; C12N 9/6427; C12N 9/6491; C12Q 1/686; C12Q 2600/158

(57) ABSTRACT

The present invention primarily relates to a method for in vitro culturing and expanding nucleus pulposus cells derived from an intervertebral disc. A secondary objective of the present invention is to provide a method for improving lower back pain by administering an effective amount of a pharmaceutical composition containing said nucleus pulposus cells obtained through the aforementioned method to subjects experiencing lower back pain.

### BACKGROUND OF THE INVENTION

### 1. Field of the Invention

The present invention relates to the technical field of cell culture and expansion methods, in particular, a method for culturing and expanding nucleus pulposus cells derived from intervertebral discs, and a method for improving lower back pain by using said nucleus pulposus cells.

### 2. Description of the Prior Art

The intervertebral disc is situated between the vertebrae and consists of the annulus fibrosus and nucleus pulposus. It serves to cushion the friction between the vertebrae during physical activities. Degenerated disc disease often leads to lower back pain, primarily affecting adults between the ages of 30 and 50. In severe cases, it can result in the collapse of the intervertebral disc. According to statistics, approximately 80% of adults have experienced lower back pain, and roughly a quarter of them suffer from back pain that hinders their work.

In general, when initial symptoms of intervertebral disc degeneration occur, clinicians will prescribe pain relief medications to temporarily alleviate patients' physical discomfort. Currently, the most commonly used medications include nonsteroidal anti-inflammatory drugs (NSAID), acetaminophen, and steroids. However, these medications can disrupt the balance of cellular secretion and may lead to gastrointestinal side effects. Severe intervertebral disc degeneration requires artificial disc replacement therapy, but artificial intervertebral disc implants are composed of polymers mixed with metals and are not biocompatible, potentially causing more serious side effects. None of the methods mentioned above can directly target degenerated discs, and there is no significant improvement in the quality of life of the patients.

When the structure of the intervertebral disc is damaged or degenerated, the nucleus pulposus tissue can become unstable and may protrude outward or even penetrate the annulus fibrosus due to increased load or pressure. This herniated nucleus pulposus tissue then exerts pressure on surrounding neural structures, such as the spinal cord or nerve roots, causing symptoms such as pain, radiating pain, numbness, or muscle weakness. Therefore, there exists a close relationship between intervertebral disc herniation and the location and pressure of the nucleus pulposus tissue.

The inventor of the present application, recognizing the limitations and drawbacks of current treatments for intervertebral disc degeneration, as well as the connection between intervertebral disc degeneration and the nucleus pulposus tissue, intends to obtain nucleus pulposus cells from the patient's intervertebral disc during the early stages of intervertebral disc degeneration. These cells are subsequently cultured, amplified in vitro, and preserved through freezing. When needed, they can be thawed, re-cultured, and transplanted into the patient's affected area, enabling them to grow without encountering immune rejection. This ultimately accomplishes the goal of regenerating nucleus pulposus tissue and effectively treating intervertebral disc degeneration. Therefore, the inventor of the present application has devoted significant effort to research and develop a method for culturing and expanding nucleus pulposus cells derived from intervertebral discs, as well as a method for using nucleus pulposus cells to improve lower back pain.

### SUMMARY OF THE INVENTION

The main object of the present invention is to provide a method for in vitro culturing and expanding nucleus pulposus cells derived from an intervertebral disc, comprising
a) providing a nucleus pulposus tissue;
b) hydrolyzing the nucleus pulposus tissue with an enzyme and obtaining primary nucleus pulposus cells by separating unhydrolyzed nucleus pulposus tissue from said primary nucleus pulposus cells through sieving and centrifugation;
c) primary culturing of said primary nucleus pulposus cells obtained in b);
d) subculturing said primary nucleus pulposus cells after said primary nucleus pulposus cells have reached a predetermined cell density during primary culture in c);
e) screening said nucleus pulposus cells from said primary nucleus pulposus cells subcultured in d) based on an expression level of one or more genes, wherein said one or more genes are selected from at least one gene module consisting of NP-development gene, NP-chondrogenic gene, NP-specific gene, NP-degeneration gene, and AF-specific gene; and
f) subjecting said nucleus pulposus cells obtained in e) to a scale-up culture.

In the method described above, the NP-development gene is KDM4E, the NP-chondrogenic gene comprises SOX9, COL2A1, and Agc1, the NP-specific gene is PAX1, the NP-degeneration gene is SAA1, and the AF-specific gene is CD90.

Furthermore, in the method described above, the enzyme used for hydrolyzing the nucleus pulposus tissue is collagenase, trypsin, or a combination thereof. When said primary nucleus pulposus cells, undergoing primary culture in step c), reach the predetermined cell density of 50-100% after 0-14 days, preferably 80-90% after 5-7 days, said primary nucleus pulposus cells are then subjected to subculturing.

Additionally, in the method described above, the expression level of said gene is determined by quantitative polymerase chain reaction (qPCR), Northern blotting, Western blotting, or DNA microarray.

Furthermore, the secondary objective of the present invention is to provide a method for improving lower back pain, comprising of administering an effective amount of a pharmaceutical composition containing said nucleus pulposus cells obtained by the method described above to a subject of lower back pain for alleviating lower back pain, wherein the pharmaceutical composition comprises a pharmaceutically acceptable carrier.

After culturing and amplifying said nucleus pulposus cells obtained from the nucleus pulposus tissue of the patient, then said nucleus pulposus cells can be re-transplanted into the affected part, specifically the degenerated part of the intervertebral disc. This transplantation is facilitated using the pharmaceutical composition containing said nucleus pulposus cells, allowing said nucleus pulposus cells to grow within the affected part. This process achieves therapeutic goals through the regeneration of nucleus pulposus tissue while minimizing the risk of immune rejection in the patient who has received the transplantation.

### BRIEF DESCRIPTION OF THE DRAWINGS

FIG. 1 shows the expression level of the NP-development gene, KDM4E, in nucleus pulposus cells from various age groups, wherein said nucleus pulposus cells were obtained by the method for in vitro culturing and expanding nucleus pulposus cells derived from an intervertebral disc as described in the present invention;

FIG. 2A shows the expression level of the NP-chondrogenic gene, SOX9, in nucleus pulposus cells from various age groups, wherein said nucleus pulposus cells were obtained by the method for in vitro culturing and expanding nucleus pulposus cells derived from an intervertebral disc as described in the present invention;

FIG. 2B shows the expression level of the NP-chondrogenic gene, COL2A1, in nucleus pulposus cells from various age groups, wherein said nucleus pulposus cells were obtained by the method for in vitro culturing and expanding nucleus pulposus cells derived from an intervertebral disc as described in the present invention;

FIG. 2C shows the expression level of the NP-chondrogenic gene, Agc1, in nucleus pulposus cells from various age groups, wherein said nucleus pulposus cells were obtained by the method for in vitro culturing and expanding nucleus pulposus cells derived from an intervertebral disc as described in the present invention;

FIG. 3 shows the expression level of the NP-specific gene, PAX1, in nucleus pulposus cells from various age groups, wherein said nucleus pulposus cells were obtained by the method for in vitro culturing and expanding nucleus pulposus cells derived from an intervertebral disc as described in the present invention;

FIG. 4 shows the expression level of the NP-degeneration gene, SAA1, in nucleus pulposus cells from various age groups, wherein said nucleus pulposus cells were obtained by the method for in vitro culturing and expanding nucleus pulposus cells derived from an intervertebral disc as described in the present invention;

FIG. 5 shows the expression level of the AF-specific gene, CD90, in nucleus pulposus cells from various age groups, wherein said nucleus pulposus cells were obtained by the method for in vitro culturing and expanding nucleus pulposus cells derived from an intervertebral disc as described in the present invention; and

FIG. 6 shows a flowchart of the method for in vitro culturing and expanding nucleus pulposus cells derived from an intervertebral disc and the method for improving lower back pain as described in the present invention.

### DETAILED DESCRIPTION OF THE PREFERRED EMBODIMENTS

Unless defined otherwise, all terms, including technical and scientific terms, used herein have the same meaning as commonly understood by a person of ordinary skill in the art to which the present inventive concepts pertain. The present invention will be further elucidated through the following exemplary embodiments; however, the present invention is not limited by these exemplary embodiments. Unless otherwise specified, the materials used in the present invention are commercially available, and the examples provided are merely illustrative of commercially available options.

### Embodiment 1: Processing Sample of Nucleus Pulposus Tissue

1. A sample of nucleus pulposus tissue from a patient with disc degeneration or disc herniation is aseptically removed during surgery in a hospital operating room. The sample of nucleus pulposus tissue is then placed in a sterile closed container containing an antibiotic composition combined with physiological saline solution and sent to a sterile laminar flow cabinet in a Good Tissue Practice (GTP) laboratory for further processing.
2. Record the weight of the nucleus pulposus tissue sample and the basic information of the patient from whom the sample was obtained.
3. Place the tissue sample in a 10 cm petri dish containing 2 mL of P0 medium (medium of passage number 0), and then cut the tissue sample into small pieces with an average size of less than 1 mm³. Approximately 5 g of tissue is used for one 10 cm petri dish, and if there is more tissue, it is divided into multiple petri dishes accordingly. The P0 medium comprises the following components: DMEM (Dulbecco's Modified Eagle Medium), human platelet lysate, and antibiotics.
4. Prepare a collagenase solution by mixing 7 mL of P0 medium with 1 mL of type I collagenase, resulting in a 7:1 volume ratio of P0 medium to type I collagenase.
5. Add the prepared collagenase solution to the petri dish containing the chopped nucleus pulposus tissue. Add 8 ml of the collagenase solution to each petri dish and incubate overnight in a cell culture incubator at 37° C. with 5% CO₂.
6. After overnight incubation, the sample of nucleus pulposus tissue from each petri dish was strained through a 100 μm cell strainer. While straining, gently grind the tissue with a 10 ml syringe plunger. After grinding, rinse the cell strainer with 10 mL of DPBS (Dulbecco's phosphate-buffered saline).
7. Centrifuge the collected cell suspension, obtained after straining, at 200×g for 5 minutes at room temperature, and then remove the supernatant after centrifugation.
8. Wash the cell pellet obtained from centrifugation twice with 20 mL of DPBS.
9. Resuspend the cell pellet in 2 mL of P0 medium and perform cell counting using trypan blue to calculate the number of cells obtained and their viability (80˜99%).
10. Plate 5˜10×10⁵ cells in a 10 cm petri dish containing 10 mL of P0 medium. At this stage, the cells are considered P0 cells (cells of passage number 0), which are the primary nucleus pulposus cells. Culture the primary nucleus pulposus cells in a cell culture incubator at 37° C. with 5% CO₂ for primary culture.
11. Continuously monitor the growth of the primary nucleus pulposus cells. On the third day, remove the old P0 medium from the petri dish, wash the primary nucleus pulposus cells once with 6 mL of DPBS, and then add 10 mL of fresh P0 medium for continued culture.
12. After 0-14 days of primary culture when the cell density reaches 50-100%, or preferably after 5-7 days when the cell density is approximately 80-90%, the primary nucleus pulposus cells can be harvested for the first subculture.

### Embodiment 2: Subculture of the Primary Nucleus Pulposus Cells

1. When the cell density of the primary nucleus pulposus cells from Embodiment 1 reaches 50˜100% or preferably 80˜90%, proceed with a subculture.
2. Remove the old P0 medium from the petri dish and wash the adherent primary nucleus pulposus cells in the 10 cm petri dish twice with 6 mL of DPBS.
3. Add 1 mL of 0.05% trypsin-EDTA (trypsin-ethylenediaminetetraacetic acid) to the 10 cm petri dish and incubate it at 37° C. for 3 minutes in a 5% CO₂ cell culture incubator.
4. After incubation, gently tap the 10 cm petri dish to detach the primary nucleus pulposus cells from the bottom of the petri dish.
5. Add 4 mL of P0 medium to the 10 cm petri dish to neutralize trypsin, and then collect the primary nucleus pulposus cells suspended in the P0 medium. Transfer the suspended primary nucleus pulposus cells into a centrifuge tube and centrifuge at 200×g for 5 minutes at room temperature.
6. After removing the supernatant from the centrifuge tube, resuspend the primary nucleus pulposus cells in an appropriate volume of culture medium. Adjust the volume of culture medium needed to resuspend the primary nucleus pulposus cells proportionally. For example, resuspend the primary nucleus pulposus cells from every 1-2 dishes of 10 cm petri dish in 1 mL of culture medium, wherein the culture medium comprises the following components: DMEM and human platelet lysate.
7. Conduct cell counting using trypan blue to determine the number of primary nucleus pulposus cells and their viability (80˜99%).

8. Subculture the resuspended primary nucleus pulposus cells in 10 ml of culture medium at a ratio of 1×10⁶ cells per 10 cm petri dish. At this stage, the primary nucleus pulposus cells are considered P1 (cells of passage number 1).
9. Conduct subculturing every 3-4 days by repeating the above steps 1-8.

### Embodiment 3: Evaluation of Gene Expression

1. During the subculturing of P2 (cells of passage number 2), P3 (cells of passage number 3), and P4 (cells of passage number 4) using the subculturing method described in Embodiment 2, collect approximately 5×10⁵ primary nucleus pulposus cells and wash them twice with 10 mL of DPBS.
2. Resuspend the collected primary nucleus pulposus cells in 1 mL of DPBS, transfer them to a centrifuge tube, and centrifuge at 200×g for 5 minutes at room temperature.
3. Remove the supernatant from the centrifuge tube, add 1 mL of GENEzol to the centrifuge tube, and store the sample containing the primary nucleus pulposus cells in the centrifuge tube at −80° C.
4. Extract RNA from the sample (primary nucleus pulposus cells) obtained in step 3 and analyze the expression levels of various genes in the primary nucleus pulposus cells using qPCR (quantitative polymerase chain reaction). The genes being analyzed are selected from the following five gene modules: I. NP-development gene (nucleus pulposus development gene), II. NP-chondrogenic gene (nucleus pulposus chondrogenic gene), III. NP-specific gene (nucleus pulposus specific gene), IV. NP-degeneration gene (nucleus pulposus degeneration gene), and V. AF-specific gene (annulus fibrosus specific gene). Specifically, the NP-development gene is KDM4E, the NP-chondrogenic gene comprises SOX9, COL2A1, and Agc1, the NP-specific gene is PAX1, the NP-degeneration gene is SAA1, and the AF-specific gene is CD90.

### Embodiment 4: Cryopreservation of Cell

1. When the primary nucleus pulposus cells are continuously cultured and expanded to P2 or P3, and the cell density reaches 80˜90%, cell cryopreservation can be performed.
2. Remove the old culture medium from the 10 cm petri dish and wash twice with 6 mL of DPBS.
3. Add 1 mL of trypsin-EDTA to the 10 cm petri dish and incubate at 37° C. for 3 minutes in a 5% CO₂ cell culture incubator.
4. After incubation, gently tap the 10 cm petri dish to detach the P2 or P3 primary nucleus pulposus cells from the bottom of the 10 cm petri dish.
5. Add 4 mL of culture medium to the 10 cm petri dish to neutralize the trypsin, then collect the P2 or P3 primary nucleus pulposus cells suspended in the culture medium. Transfer the primary nucleus pulposus cells to a centrifuge tube and centrifuge at 200×g for 5 minutes at room temperature.
6. After removing the supernatant from the centrifuge tube, resuspend the P2 or P3 primary nucleus pulposus cells in a culture medium of appropriate volume. Adjust the volume of culture medium required to resuspend the P2 or P3 primary nucleus pulposus cells proportionally. Specifically, resuspend the P2 or P3 primary nucleus pulposus cells from every 1 to 2 10 cm petri dishes in 1 mL of culture medium.
7. Perform cell counting with trypan blue to confirm the number of P2 or P3 primary nucleus pulposus cells and their viability (80-99%).
8. Centrifuge the P2 or P3 primary nucleus pulposus cells resuspended in culture medium in a centrifuge tube at 200×g for 5 minutes at room temperature, then remove the supernatant.
9. Resuspend the P2 or P3 primary nucleus pulposus cells in an appropriate volume of cell freezing medium at a cell concentration of 2×10⁶/ml.
10. Dispense the suspension of P2 or P3 primary nucleus pulposus cells into cryovials at a proportion of 1 mL/vial.
11. Place the cryovials into a cryo freezing container, and then store the cryo freezing container in a −80° C. freezer. Transfer the cryovials to liquid nitrogen the next day for storage.

Table 1 below shows the sources of reagents used in the above-mentioned embodiments.

TABLE 1

| | | | |
|---|---|---|---|
| | Company | Product | |
| Product name | name | number | Content |

1. Basal medium

| | | | |
|---|---|---|---|
| DMEM | GENEDireX | CC103-0500 | Sodium Pyruvate |
| (Dulbecco's | | | Glutamine |
| Modified Eagle | | | Phenol Red |
| Medium), High | | | |
| Glucose | | | |
| DMEM, High | Gibco | 11995-040 | Sodium Pyruvate |
| Glucose | | | Glutamine |
| | | | Phenol Red |

2. Serum

| | | |
|---|---|---|
| Human Platelet | EliteCell | EPA-500 |
| Lysate | | |
| Human Platelet | EliteCell | EPAGMP -500 |
| Lysate | | |

3. Antibiotics

| | | | |
|---|---|---|---|
| Antibiotic | cellgro | 30004141 | penicillin G, |
| Antimycotic | | | streptomycin |
| | | | sulfate, |
| | | | amphotericin B |
| Antibiotic | Gibco | 15240-062 | penicillin G, |
| Antimycotic | | | streptomycin |
| | | | sulfate, |
| | | | amphotericin B |

4. Buffer solution

| | | | |
|---|---|---|---|
| PBS | Omics bio | IB3012 | NaCl, KCl, |
| | | | KH₂PO₄, |
| | | | Na₂HPO₄ |
| DPBS | Gibco | 14190-0144 | NaCl, KCl, |
| | | | KH₂PO₄, |
| | | | Na₂HPO₄ |

5. Enzyme

| | | |
|---|---|---|
| 2.5% Trypsin | cellgro | 27419007 |
| Collagenase I | SIGMA | 9001-12-1 |
| Trypsin | Gibco | 25300-054 |

6. Cell freezing medium

| | | |
|---|---|---|
| BAMBANKER | GC LYMPHOTEC | BB01 |
| CryoStor CS10 | Biolife | 210102 |

7. Dye

| | | |
|---|---|---|
| trypan blue | SIGMA | 72-57-1 |

### Embodiment 5: Expression Level of NP-development Gene (KDM4E) in Primary Nucleus Pulposus Cells

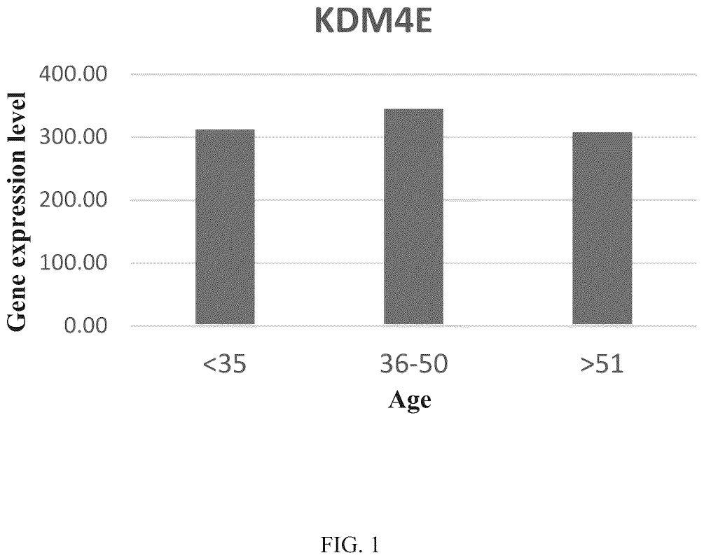

FIG. 1 shows the results of the analysis of the expression levels of the NP-development gene (KDM4E) in primary nucleus pulposus cells obtained through the culturing and expanding methods described in Embodiments 1 to 2, across different age groups. This analysis was conducted using the gene expression evaluation method described in Embodiment 3. As shown in the bar chart in FIG. 1, primary nucleus pulposus cells obtained from individuals under 35 years old and those aged 36-50 exhibited higher expression levels of the NP-development gene KDM4E compared to individuals over 51 years old. Moreover, the specific expression of the NP-development gene KDM4E confirms that the primary nucleus pulposus cells obtained through the culturing and expanding method of the present invention in Embodiments 1 and 2 are indeed generated through the process of nucleus pulpous development.

### Embodiment 6: Expression Level of NP-chondrogenic Gene (SOX9, COL2A1 and Agc1) in Primary Nucleus Pulposus Cells

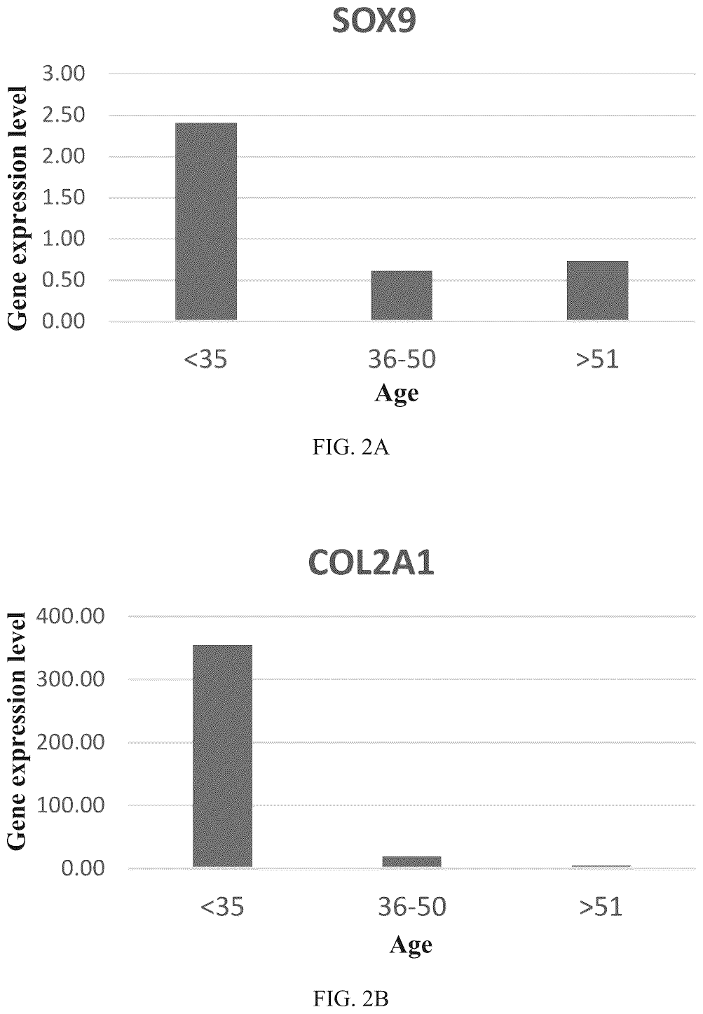

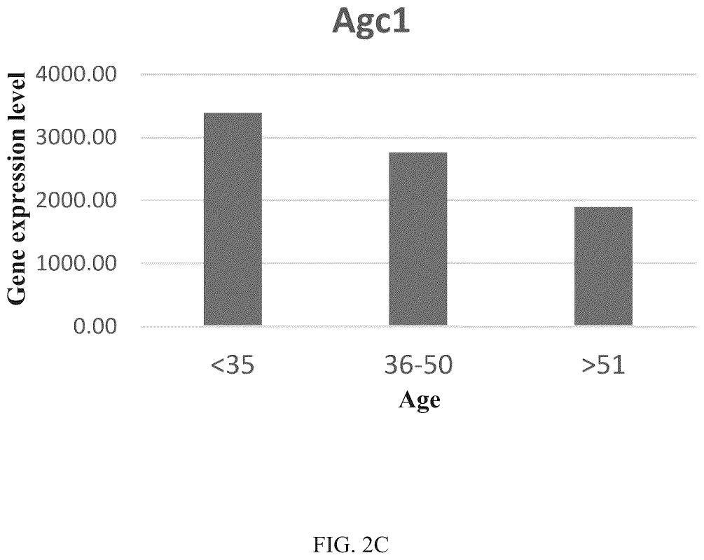

FIGS. 2A to 2C show the results of the analysis of the expression levels of the NP-chondrogenic gene (SOX9, COL2A1, and Agc1) in primary nucleus pulposus cells obtained through the culturing and expanding methods described in Embodiments 1 to 2, across different age groups. This analysis was conducted using the gene expression evaluation method described in Embodiment 3. As shown in the bar chart in FIG. 2A, primary nucleus pulposus cells obtained from individuals under 35 years old exhibited significantly higher expression levels of the NP-chondrogenic gene SOX9 compared to those in the age groups of 36-50 and over 51 years old. SOX9 is an important early transcription factor in chondrogenic differentiation. As shown in the bar chart in FIG. 2B, primary nucleus pulposus cells obtained from individuals under 35 years old and those aged 36-50 had higher expression levels of the NP-chondrogenic gene COL2A1 compared to individuals over 51 years old. Notably, the expression level of COL2A 1 in individuals under 35 years old was also significantly higher than in the 36-50 and over 51 years old age groups. COL2A1 is an important gene for the cartilage matrix in chondrogenic differentiation. As shown in the bar chart in FIG. 2C, the expression levels of the NP-chondrogenic gene Agcl in primary nucleus pulposus cells obtained through the culturing and expanding methods of the present invention varied with age. Specifically, individuals under 35 years old had the highest expression, followed by those aged 36-50, and individuals over 51 years old had the lowest expression. Agcl is an important gene for the cartilage matrix in chondrogenic differentiation. Furthermore, from FIGS. 2A to 2C, it can be seen that primary nucleus pulposus cells obtained through the culturing and expanding methods described in Embodiments 1 to 2 of the present invention all exhibit the expression of important NP-chondrogenic gene associated with nucleus pulposus tissue. Additionally, their expression levels decrease with increasing age.

### Embodiment 7: Expression Level of NP-Specific Gene (PAX1) in Primary Nucleus Pulposus Cells

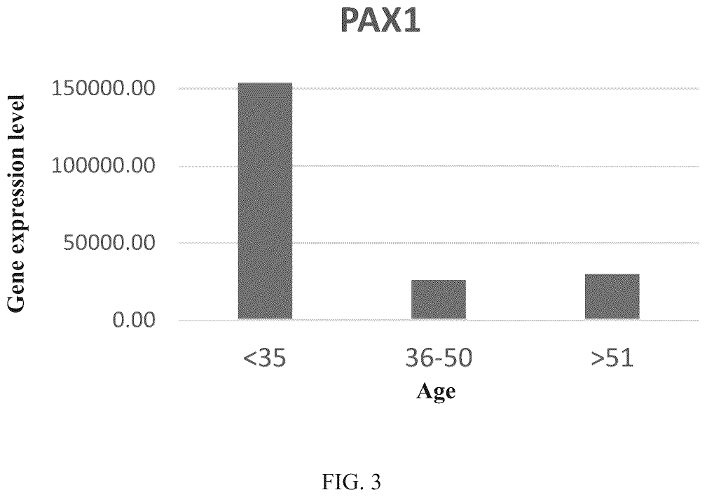

FIG. 3 shows the results of the analysis of the expression levels of the NP-specific gene (PAX1) in primary nucleus pulposus cells obtained through the culturing and expanding methods described in Embodiments 1 to 2, across different age groups. This analysis was conducted using the gene expression evaluation method described in Embodiment 3. As shown in the bar chart in FIG. 3, primary nucleus pulposus cells obtained from individuals under 35 years old exhibited significantly higher expression levels of the NP-specific gene PAX1 compared to those in the age groups of 36-50 and over 51 years old. PAX1 is a precursor gene for nucleus pulposus tissue, and in younger individuals, the expression levels of the NP-specific gene PAX1 are higher. Moreover, a higher expression level of the NP-specific gene PAX1 implies a greater potential for nucleus pulposus tissue repair. Therefore, FIG. 3 demonstrates that primary nucleus pulposus cells obtained through the culturing and expanding methods described in Embodiments 1 to 2 of the present invention all exhibit the expression of the PAX1 gene associated with the repair capability of nucleus pulposus tissue. Notably, their expression levels are substantially higher in younger individuals.

### Embodiment 8: Expression Level of NP-Degeneration Gene (SAA1) in Primary Nucleus Pulposus Cells

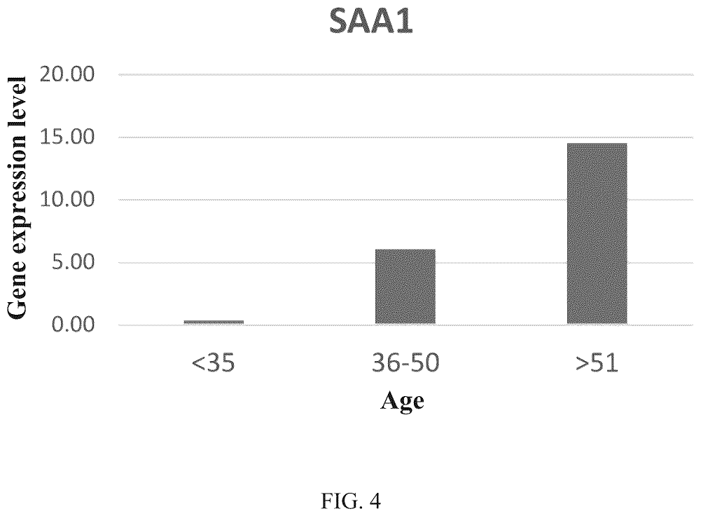

FIG. 4 shows the results of the analysis of the expression levels of the NP-degeneration gene (SAA1) in primary nucleus pulposus cells obtained through the culturing and expanding methods described in Embodiments 1 to 2, across different age groups. This analysis was conducted using the gene expression evaluation method described in Embodiment 3. As shown in the bar chart in FIG. 4, the expression levels of the NP-degeneration gene SAA1 in primary nucleus pulposus cells obtained through the culturing and expanding methods of the present invention varied with age. Specifically, individuals over 51 years old exhibited the highest expression, followed by those in the 36-50 years age group, while individuals under 35 years old had the lowest expression. Notably, SAA1 is expressed during nucleus pulposus degeneration and nucleus pulposus cell apoptosis, with its expression increasing with age. Therefore, FIG. 4 demonstrates that primary nucleus pulposus cells obtained through the culturing and expanding methods described in Embodiments 1 to 2 of the present invention exhibit higher expression levels of the NP-degeneration gene SAA1 as age increases. In other words, the primary nucleus pulposus cells cultured and expanded using the methods of the present invention exhibit characteristics similar to those expressed by cells of nucleus pulposus tissue.

### Embodiment 9: Expression Level of AF-Specific Gene (CD90) in Primary Nucleus Pulposus Cells

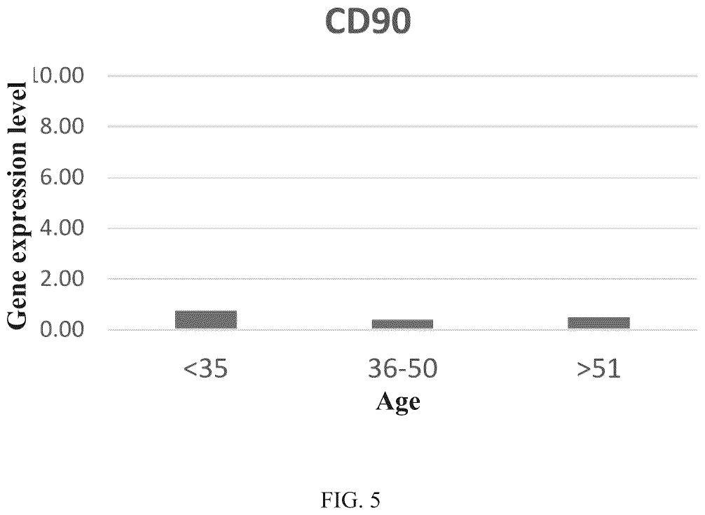

FIG. 5 shows the results of the analysis of the expression levels of the AF-specific gene (CD90) in primary nucleus pulposus cells obtained through the culturing and expanding methods described in Embodiments 1 to 2, across different age groups. This analysis was conducted using the gene expression evaluation method described in Embodiment 3. As shown in the bar chart in FIG. 5, the expression levels of the AF-specific gene CD90 in primary nucleus pulposus cells obtained through the culturing and expanding methods of the present invention were very low across different age groups. In other words, the markedly low expression levels of the AF-specific gene CD90 serve as a form of reverse validation, suggesting that primary nucleus pulposus cells obtained through the culturing and expanding methods of the present invention do not originate from the annulus fibrosus.

Table 2 below presents the results of the analysis of gene expression levels in primary nucleus pulposus cells obtained through the culturing and expanding methods described in Embodiments 1 to 2, across different age groups. This analysis encompassed five gene modules: NP-development gene, NP-chondrogenic gene, NP-specific gene, NP-degeneration gene, and AF-specific gene. The gene expression evaluation method described in Embodiment 3 was employed for this analysis.

TABLE 2

| | | I | | | | III | IV | V |
|---|---|---|---|---|---|---|---|---|
| | | NP- | II | | | NP- | NP- | AF- |
| Genes Definition | | development | NP-chondrogenic | | | specific | degeneration | specific |
| Ages (yo) | Donor | KDM4E | SOX9 | COL2A1 | Agc1 | PAX1 | SAA1 | CD90 |
| <35 | 1 | 293.26 | 0.50 | 124.61 | 1604.55 | 11170.19 | 0.41 | 0.44 |
| | 2 | 237.12 | 2.98 | 42.56 | 1977.95 | 239344.10 | 0.59 | 1.46 |
| | 3 | 668.80 | 2.17 | 1009.97 | 3019.79 | 128443.34 | 0.13 | 0.52 |
| | 4 | 49.11 | 3.95 | 243.34 | 6951.37 | 235796.90 | 0.17 | 0.56 |
| AVG | | 312.07 | 2.40 | 355.12 | 3388.42 | 153688.63 | 0.33 | 0.75 |
| 36-50 | 1 | 731.66 | 0.31 | 10.38 | 1262.03 | 29458.52 | 0.60 | 0.30 |
| | 2 | 247.70 | 1.03 | 24.20 | 6682.70 | 27977.75 | 0.50 | 0.70 |
| | 3 | 55.10 | 0.49 | 21.41 | 327.34 | 19964.66 | 16.96 | 0.20 |
| AVG | | 344.82 | 0.61 | 18.66 | 2757.36 | 25800.31 | 6.02 | 0.40 |
| >51 | 1 | 641.55 | 0.54 | 0.2 | 227.86 | 8606.49 | 0.01 | 0.43 |
| | 2 | 252.32 | 1.2 | 1.33 | 5260.34 | 53383.61 | 1.21 | 0.8 |
| | 3 | 29.72 | 0.45 | 12.26 | 180.48 | 27815.83 | 42.31 | 0.24 |

| | | | | | | | |
|---|---|---|---|---|---|---|---|
| AVG | 307.86 | 0.73 | 4.60 | 1889.56 | 29935.31 | 14.51 | 0.49 |

The data presented in Table 2 corresponds to the information depicted in the bar charts of FIGS. 1 to 5. These data represent the average values of gene expression levels obtained in various age groups. When combining the results from FIGS. 1 to 5 with the data in Table 2, it becomes evident that through the method provided by the present invention for in vitro culturing and expanding nucleus pulposus cells derived from an intervertebral disc, along with the gene expression evaluation method using five gene modules: NP-development gene, NP-chondrogenic gene, NP-specific gene, NP-degeneration gene, and AF-specific gene, it is possible to obtain nucleus pulposus cells with the ability to repair the function of nucleus pulposus tissue. Subsequently, these nucleus pulposus cells can be used to produce a pharmaceutical composition for the treatment of lower back pain stemming from chronic lumbar pain due to intervertebral disc degeneration, through the application of the pharmaceutical composition containing these nucleus pulposus cells.

### Embodiment 10: Flowchart of the Method for Culturing and Expanding

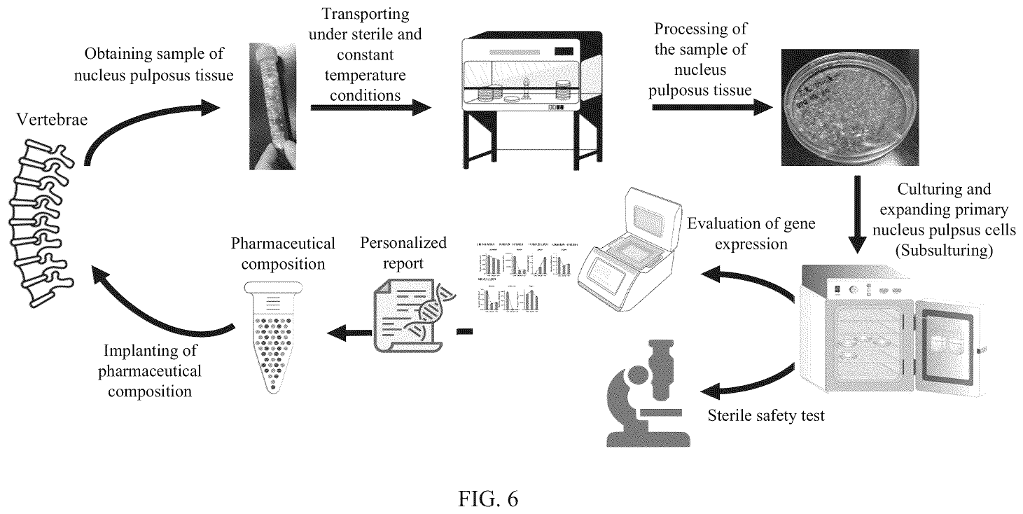

Nucleus Pulposus Cells Derived from Intervertebral Discs and Its Application FIG. 6 illustrates a flowchart of the method for culturing and expanding nucleus pulposus cells derived from intervertebral discs, as well as its application, according to the present invention. Firstly, as described in Embodiment 1, a sample of nucleus pulposus tissue is aseptically obtained from a patient suffering from intervertebral disc degeneration or disc herniation through a surgical procedure. Subsequently, the sample of nucleus pulposus tissue is transported under sterile, constant temperature conditions to a sterile laminar flow cabinet within a Good Tissue Practice (GTP) laboratory for further processing, resulting in the generation of primary nucleus pulposus cells. Following this, the primary nucleus pulposus cells obtained, as described in Embodiment 1, undergo subculture and amplification using the method detailed in Embodiment 2. Then, the gene expression evaluation method described in Embodiment 3 is employed to assess these primary nucleus pulposus cells. Simultaneously, a sterile safety test is conducted to confirm that these primary nucleus pulposus cells remain uncontaminated, such as by mycoplasma contamination. Based on the results of the gene expression evaluation and sterile safety test, a personalized report is prepared to determine whether the primary nucleus pulposus cells obtained from the patient's nucleus pulposus tissue possess regenerative capabilities and exhibit characteristics of nucleus pulposus cells. Using this report, nucleus pulposus cells exhibiting regenerative capabilities and characteristics of nucleus pulposus cells are selected for the customized preparation of a pharmaceutical composition to treat lower back pain (intervertebral disc degeneration or disc herniation) specific to the patient. Ultimately, the pharmaceutical composition is implanted in the patient's affected area, allowing the nucleus pulposus cells with regenerative capabilities to grow at the affected area and achieve the goal of treating intervertebral disc degeneration or disc herniation by regenerating nucleus pulposus tissue.

The above descriptions have comprehensively introduced the method for culturing and expanding nucleus pulposus cells derived from intervertebral discs and for alleviating lower back pain using nucleus pulposus cells according to the present invention. It should be emphasized that the above descriptions are made on embodiments of the present invention; however, the embodiments are not intended to limit the scope of the present invention, and all equivalent implementations or alterations within the spirit of the present invention still fall within the scope of the present invention.

What is claimed is:

1. A method for in vitro culturing and expanding nucleus pulposus cells derived from an intervertebral disc, comprising
a) providing a nucleus pulposus tissue;
b) hydrolyzing the nucleus pulposus tissue with an enzyme and obtaining primary nucleus pulposus cells by separating unhydrolyzed nucleus pulposus tissue from said primary nucleus pulposus cells through sieving and centrifugation;
c) primary culturing of said primary nucleus pulposus cells obtained in b);
d) subculturing said primary nucleus pulposus cells after said primary nucleus pulposus cells have reached a predetermined cell density during primary culture in c);
e) screening said nucleus pulposus cells from said primary nucleus pulposus cells subcultured in d) based on an expression level of one or more genes, wherein said one or more genes are selected from at least one gene module consisting of NP-development gene, NP-chondrogenic gene, NP-specific gene, NP-degeneration gene, and AF-specific gene; and
f) subjecting said nucleus pulposus cells obtained in e) to a scale-up culture.

2. The method of claim 1, wherein the NP-development gene is KDM4E.

3. The method of claim 1, wherein the NP-chondrogenic gene comprises SOX9, COL2A1, and Agc1.

4. The method of claim 1, wherein the NP-specific gene is PAX1.

5. The method of claim 1, wherein the NP-degeneration gene is SAA1.

6. The method of claim 1, wherein the AF-specific gene is CD90.

7. The method of claim 1, wherein the enzyme for hydrolyzing the nucleus pulposus tissue is collagenase, trypsin, or a combination thereof.

8. The method of claim 1, wherein the predetermined cell density is a cell density of 80 to 90%.

9. The method of claim 1, wherein the expression level of said gene is determined by quantitative polymerase chain reaction (qPCR), Northern blotting, Western blotting, or DNA microarray.

10. A method for improving lower back pain, comprising of administering an effective amount of a pharmaceutical composition containing said nucleus pulposus cells obtained by the method of claim 1 to a subject of lower back pain for alleviating lower back pain.
